(19) United States
(12) Patent Application Publication
(10) Pub. No.: US 2026/0207838 A1
(43) Pub. Date: Jul. 23, 2026

(54) SIGNAL NOISE REDUCTION FOR FLUID DELIVERY SYSTEM

(71) Applicant: CareFusion 303, Inc., San Diego, CA (US)

(72) Inventors: Likhith NALLURI, Brea, CA (US); Tomas FRAUSTO, Walnut, CA (US); Eugene E. MASON, La Habra Heights, CA (US)

(21) Appl. No.: 19/035,217

(22) Filed: Jan. 23, 2025

(51) Int. Cl. A61M 5/14; A61M 5/168; A61M 39/10

(52) U.S. Cl. CPC A61M 5/1414; A61M 5/16854; A61M 39/10; A61M 2039/1027; A61M 2202/0007; A61M 2202/04; A61M 2205/3344

(57) ABSTRACT

In order to accurately monitor fluid flow through an infusion line, it can be desirable to position a pressure transducer along the infusion line and a noise-filtering mechanism between the pressure transducer and the fluid. Fluid in the infusion line transmits a pressure wave that is partially transmitted through the noise-filtering mechanism, such that the parts of the pressure wave with high amplitudes are received by the pressure transducer and the rest of the pressure wave (i.e., the noise) is reflected back towards the fluid. The noise-filtering fluid measurement system disclosed herein utilizes a layered structure of gel and air to gradually change the impedance of the materials through which a pressure wave must travel to ensure that only the meaningful parts of the pressure wave reach the pressure transducer while also preventing the entirety of the pressure wave from being reflected back towards the fluid.

### TECHNICAL FIELD

The present invention relates generally to fluid delivery devices and systems and, in particular, to a fluid delivery measurement device that uses a pressure transducer to monitor fluid delivery to a patient.

### BACKGROUND

Medical treatments often include continuous, repeated, or automated fluid delivery. Examples of such fluid delivery include the infusion of a medical fluid (e.g., a saline solution or a liquid medication) to patients using an intravenous (IV) catheter, patient-controlled analgesia, anesthesia, chemotherapy, insulin pumps, neonatal care, and intravenous rehydration.

In situations where a patient might need to receive medication through continuous, repeated, or automated infusion, fluid can be stored in an infusion pump, IV bag, or other fluid source. The fluid delivery system is generally set up to deliver a precise amount of fluid to the patient over a specific period of time.

It can be desirable to use pressure transducers or pressure sensors to monitor backpressure in the infusion line. For example, the pressure transducer can provide a continuous feedback mechanism that ensures that the fluid is delivered at the correct rate and at the correct volume, which makes the fluid delivery safer and more accurate. The pressure transducer can also be used to alert caregivers to occlusions in the infusion line, which means the occlusions can be addressed by caregivers in a timelier fashion—thus reducing the negative consequences associated with occlusions.

### SUMMARY

The quantity and speed of fluid delivered to a patient through an infusion line can be monitored by a pressure transducer or a pressure sensor positioned along the infusion line in between the fluid source (e.g., an IV bag) and the patient. However, there is a challenge in using pressure transducers to monitor fluid flow through the infusion line because the continuous flow of fluid past the pressure transducer produces a signal with a lot of noise. The noise in the signal can make it difficult for caregivers (or other medical fluid administrators) to identify issues in the fluid flow to the patient (e.g., occlusions in the infusion line). Therefore, a signal-reducing or noise-filtering fluid delivery measurement system or desirable for producing clean and easily comprehendible fluid delivery data.

This effect can be accomplished by positioning a noise-filtering mechanism between the fluid in the infusion line and the pressure transducer. Specifically, this can be accomplished by positioning materials with a variety of impedances between the fluid and the pressure transducer because the different impedances of the different materials will ensure that only part of the pressure wave (e.g., the parts with the highest amplitude) are received by the pressure transducer.

Accordingly, some embodiments of the present disclosure are directed to a noise-filtering fluid delivery tube, the tube comprising: a delivery portion comprising two ends and a lateral opening between the two ends, wherein the delivery portion is configured to transport fluid from a fluid source to a patient; and a noise-filtering portion that is transverse to the delivery portion and intersects the delivery portion at the lateral opening, wherein the noise-filtering portion comprises a first gel layer adjacent to the lateral opening, a second gel layer adjacent to a distal opening opposite the lateral opening, and an air pocket between the first and second gel layers.

In some embodiments of the noise-filtering fluid delivery tube, the delivery portion and the noise-filtering portion each comprise a hollow cylinder, the noise-filtering portion being shorter than the delivery portion.

In some embodiments of the noise-filtering fluid delivery tube, each of the two ends comprises a needleless fluid connector. Optionally, each of the needleless fluid connectors comprises a male luer lock or a female luer lock.

In some embodiments of the noise-filtering fluid delivery tube, the lateral opening comprises a lateral membrane adjacent to the first gel layer, the lateral membrane being configured to separate the fluid in the delivery portion from the first gel layer.

In some embodiments of the noise-filtering fluid delivery tube, the distal opening of the noise-filtering portion is configured to couple with a pressure transducer. Optionally, the distal opening comprises a distal membrane adjacent to the second gel layer, wherein the distal membrane is configured to separate the second gel layer from the pressure transducer. Optionally, the second gel layer of the noise-filtering portion is configured to surround the pressure transducer. Optionally, the first gel layer is configured to separate the fluid from the air pocket, and the second gel layer is configured to separate the air pocket from the pressure transducer.

In some embodiments of the noise-filtering fluid delivery tube, gel in the first and second gel layers comprises a first impedance and air in the air pocket comprises a second impedance, the first impedance being greater than the second impedance, but less than an impedance of the fluid being transported through the delivery portion.

Other embodiments of the present disclosure are directed to a noise-filtering fluid delivery tube comprising: a delivery portion configured to fluidly connect a fluid source and a patient; and a noise-filtering portion transverse to the delivery portion, the noise-filtering portion comprising a gel filling and an air pocket positioned within the gel filling.

In some embodiments of the noise-filtering fluid delivery tube, the gel filling comprises a first gel layer adjacent to the delivery portion, and the tube further comprises a first membrane between the first gel layer and the delivery portion.

In some embodiments of the noise-filtering fluid delivery tube, the gel filling is configured to abut a pressure transducer. Optionally, the gel filling comprises a second gel layer that is configured to abut the pressure transducer, and the noise-filtering portion further comprises a second membrane configured to separate the second gel layer from the pressure transducer.

In some embodiments of the noise-filtering fluid delivery tube, the gel filling is configured to surround a pressure transducer.

Other embodiments of the present disclosure are directed to a noise-filtering fluid measurement system comprising: a fluid delivery tube comprising: a delivery portion configured to transport fluid from a fluid source to a patient, wherein the delivery portion extends along a first longitudinal axis and comprises a lateral opening; and a noise-filtering portion extending from the delivery portion at the lateral opening along a second longitudinal axis that is transverse to the first longitudinal axis, wherein the noise-filtering portion comprises a distal opening opposite the lateral opening, a gel filling between the lateral and distal openings, and an air pocket within the gel filling; a mount comprising a tube engagement feature, wherein the fluid delivery tube is coupled to the mount via the tube engagement feature; and a pressure transducer coupled to the mount, wherein the pressure transducer is aligned with the second longitudinal axis of the noise-filtering portion.

In some embodiments of the noise-filtering fluid measurement system, the gel filling surrounds the pressure transducer.

In some embodiments of the noise-filtering fluid measurement system, the gel filling comprises a first gel layer in between the lateral opening and the air pocket and a second gel layer in between the air pocket and the distal opening.

In some embodiments of the noise-filtering fluid measurement system, gel in the gel filling comprises a first impedance and air in the air pocket comprises a second impedance, the first impedance being greater than the second impedance, but less than an impedance of the fluid being transported through the delivery portion.

In some embodiments of the noise-filtering fluid measurement system, the tube engagement feature comprises one or more of a channel, a plurality of clamps, and a cavity.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set 1 coupled to a patient, in accordance with some embodiments described herein.

FIGS. 2A and 2B illustrate perspective views of the noise-filtering fluid measurement system and the noise-filtering fluid delivery tube, in accordance with some embodiments described herein.

FIGS. 3A and 3B illustrate cross-sectional views of two embodiments of the noise-filtering fluid measurement system, in accordance with some embodiments described herein.

FIGS. 4A and 4B illustrate additional embodiments of the mount of the noise-filtering fluid measurement system, in accordance with some embodiments described herein.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Monitoring fluid flow through an infusion line is desirable in situations where fluid is being delivered to a patient continuously, repeatedly, or automatically. Fluid flow can be monitored using a pressure transducer that is positioned along the infusion line. As the fluid flows past the pressure transducer, the fluid transmits pressure waves. The pressure transducer receives the pressure waves and produces a signal that can be used to identify the quantity and rate of fluid (to name two examples) delivered to the patient. The continuous flow of fluid can result in the pressure transducer producing a very noisy signal that is difficult for caregivers to read. Therefore, reducing the amount of noise in the signal produced by the pressure transducer can be valuable. This can be accomplished with a device that includes materials of various impedances that the pressure wave must travel through before reaching the pressure transducer.

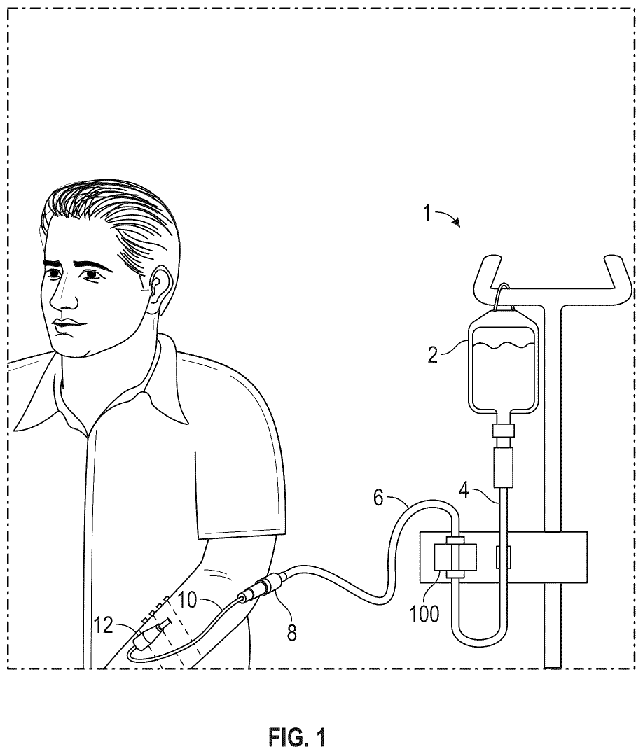

Referring now to the figures, FIG. 1 illustrates an IV set 1 coupled to a patient, in accordance with some embodiments described herein. The IV set 1 includes a medical fluid bag 2 (which can also be referred to as an IV bag 2 or a fluid source 2) filled with fluid such as saline or medication. The fluid from the medical fluid bag 2 travels through the first tubing 4, the noise-filtering fluid measurement system 100, the second tubing 6, the fluid connector 8, the third tubing 10, and the catheter 12 into a vein in the patient's arm. The noise-filtering fluid measurement system 100 includes a pressure transducer that measures fluid flow through the first and second tubings 4, 6, and, thus, the fluid delivered to the patient from the medical fluid bag 2.

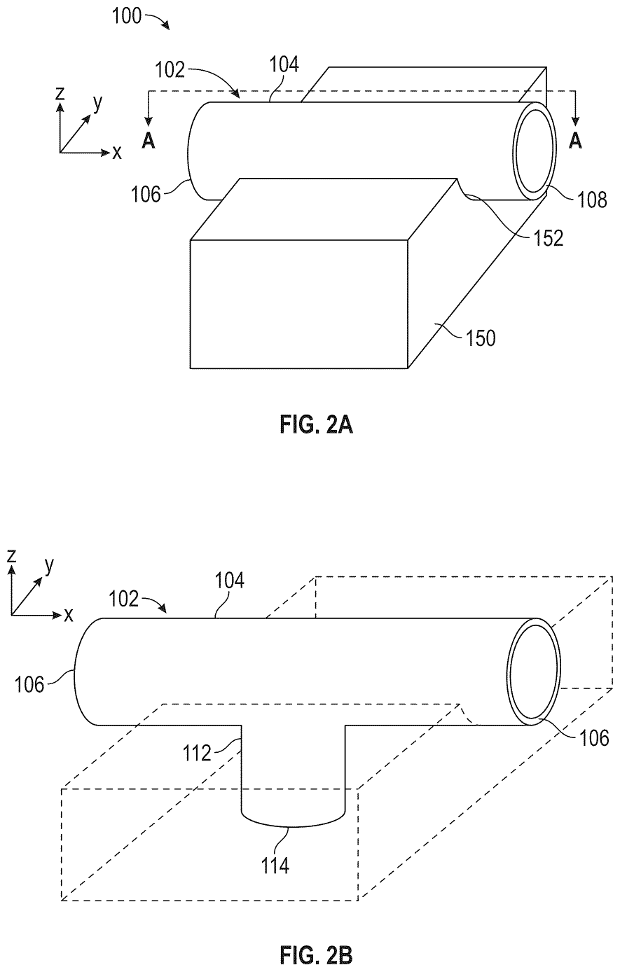

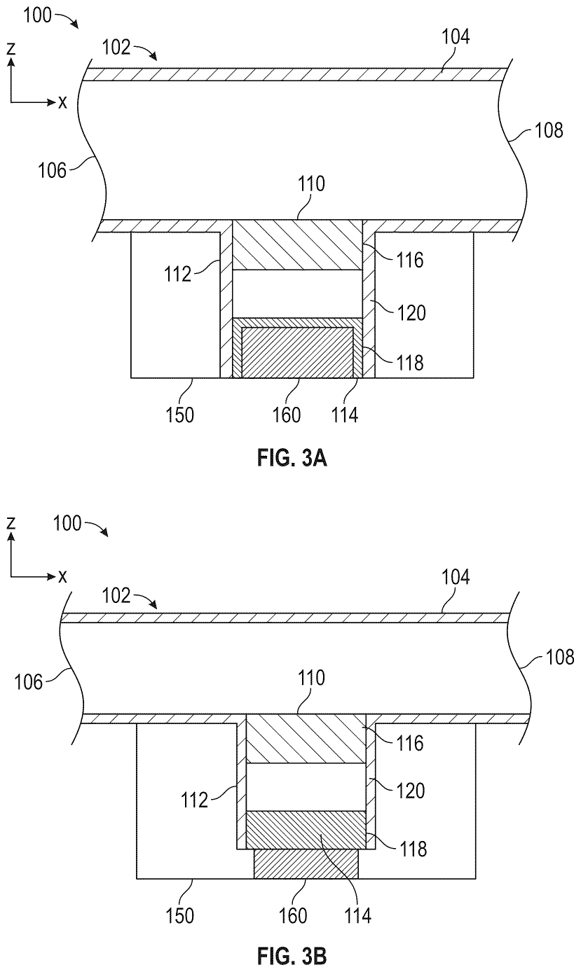

FIGS. 2A and 2B illustrate perspective views of the noise-filtering fluid measurement system and the noise-filtering fluid delivery tube, in accordance with some embodiments described herein. The noise-filtering fluid measurement system 100 is made up of a mount 150 that holds a noise-filtering fluid delivery tube 102 (which can also be referred to as a tube 102) and a pressure transducer (e.g., the pressure transducer 160 shown in FIGS. 3A-3B). The tube 102 has two portions: a delivery portion 104 that transports fluid from a fluid source (e.g., the IV bag 2 in FIG. 1) to a patient and a noise-filtering portion 112 that filters the signal sent by the fluid to the pressure transducer. The pressure transducer measures the fluid flowing through the delivery portion 104 by receiving pressure waves transmitted by the fluid. The pressure waves travel through the noise-filtering portion 112 before reaching the pressure transducer. The noise-filtering portion 112 contains multiple mediums, and the change in medium reduces noise in the signal produced by the pressure transducer. The multiple mediums of the noise-filtering portion 112 and their signal-reducing effect are described in greater detail below.

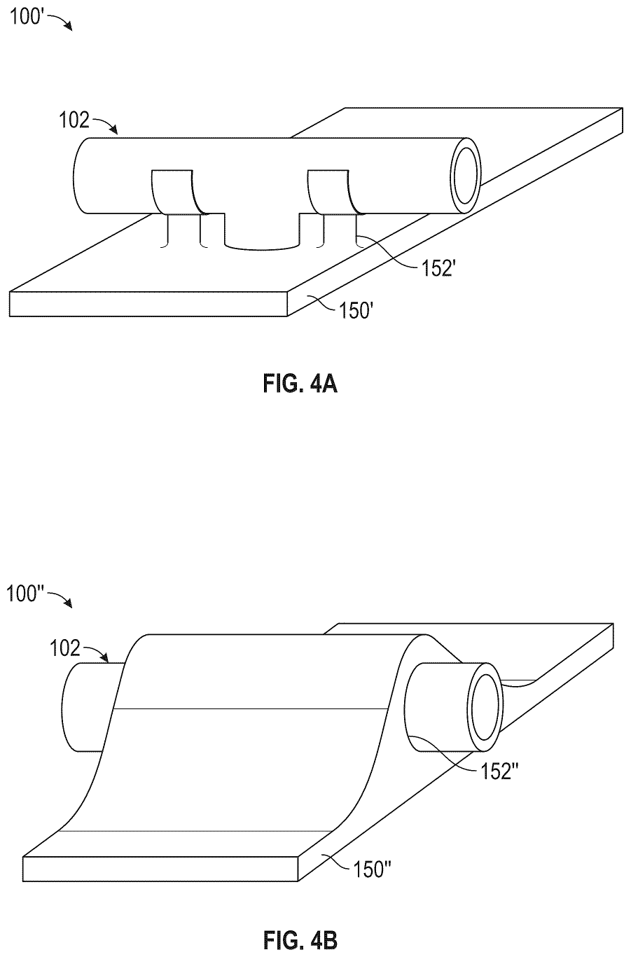

The mount 150 can be anchored to the patient (e.g., taped or strapped to the patient's arm) or an IV pole. Other positions and anchoring configurations are also possible. Essentially, the mount 150 holds the tube 102 at a position between the fluid source and the patient along the tubing of the IV set (e.g., between the first and second tubings 4, 6 in the IV set 1 shown in FIG. 1) so that the fluid can travel from the fluid source, through the tube 102, and to the patient. The mount 150 includes an engagement feature 152 by which the tube 102 is secured to the mount 150 (and, thus, coupled to the IV set and the patient). In the embodiment shown in FIGS. 2A-2B, the engagement feature 152 is made of two transverse channels. The channel that extends along the x-axis supports the delivery portion 104 of the tube 102, and the channel that extends along the z-axis holds the noise-filtering portion 112 of the tube 102. Other embodiments of the engagement feature 152 are shown in FIGS. 4A-4B and described in greater detail below.

The mount 150 also includes the pressure transducer and couples the tube 102 to the pressure transducer. The pressure transducer measures the pressure of the fluid as it flows through the infusion line (i.e., the first and second tubings 4, 6 shown in FIG. 1). Specifically, as the fluid flows through the infusion line and past the pressure transducer, the fluid transmits pressure waves and applies forces to the pressure transducer. The pressure transducer receives the pressure waves and measures the applied forces, and this measurement is converted to indicate the amount of fluid that is delivered to the patient. This measurement can also be used to identify occlusions in the infusion lines by identifying changes in the force being applied to the pressure transducer. The positioning of the pressure transducer relative to the tube 102, particularly the noise-filtering portion 112, is described in greater detail below with respect to FIGS. 3A-3B.

As mentioned above, the noise-filtering delivery tube 102 has a delivery portion 104 and a noise-filtering portion 112. Both the delivery portion 104 and the noise-filtering portion 112 are hollow cylinders, with the delivery portion 104 generally being longer (and, thus, suited to transport fluid along the infusion line) than the noise-filtering portion (which generally is shorter than the delivery portion 104 because the pressure waves from the fluid have to travel through the noise-filtering portion to reach the pressure transducer). The delivery portion 104 extends along a first longitudinal axis and has two ends (a first end 106 and a second end 108) and a lateral opening (e.g., lateral opening 110 shown in FIGS. 3A-3B). Each of the two ends 106, 108, which are located along the first longitudinal axis, has a fluid connector (e.g., a needleless fluid connector) so that the tube 102 can be fluidly connected to the infusion line. For example, the first end 106 can have a male luer lock that is couples to a female luer lock of an extension tube (such as first tubing 4 of FIG. 1). Similarly, the second end 108 can have a female luer lock that couples to a male luer lock of an extension tube (such as second tubing 6 of FIG. 1). Other types of fluid connectors are other possible. It is also possible for the first and second ends 106, 108 of the delivery portion 104 to have the same type of fluid connector.

The noise-filtering portion 112 of the tube 102 extends from the delivery portion 104 at the lateral opening of the delivery portion 104 along a second longitudinal axis that is transverse to the first longitudinal axis. The noise-filtering portion 112 also has a distal opening 114. Both the lateral opening and the distal opening 114 are positioned along the second longitudinal axis of the noise-filtering portion, with the lateral opening being at a top end of the noise-filtering portion 112 (which connects to the delivery portion 104) and the distal opening 114 being at a bottom end of the noise-filtering portion 112. The bottom end of the noise-filtering portion 112 is near the bottom (or base) portion of the mount 150. In some embodiments, the noise-filtering portion 112 is flush-mounted to the base portion of the mount 150 via the distal opening 114. In the embodiment shown in FIGS. 2A-2B, the distal opening 114 sits at the bottom of the engagement feature 152—specifically, the bottom of the channel that extends along the z-axis.

FIGS. 3A and 3B illustrate cross-sectional views of two embodiments of the noise-filtering fluid measurement system, in accordance with some embodiments described herein. In each of FIGS. 3A and 3B, the cross-section is taken along the line A-A in FIG. 2A. Both FIGS. 3A and 3B show that the noise-filtering fluid measurement system 100 has noise-filtering fluid delivery tube 102 positioned inside a mount 150. The delivery portion 104 of the tube 102 has two ends 106, 108 and a lateral opening 110. The lateral opening 110 is on the curved surface (or the lateral portion) of the hollow cylinder that makes up the delivery portion 104. The lateral opening 110 is also on the upper circular surface (or the proximal side) of the hollow cylinder that makes up the noise-filtering portion 112. As shown, the noise-filtering portion 112 is transverse to the delivery portion 104 and intersects the delivery portion 104 at the lateral opening 110.

The noise-filtering portion 112 has an air pocket 120 surrounded by gel 116, 118. Because of the air pocket 120, pressure waves that are transmitted by the fluid in the delivery portion 104 are received by the pressure transducer 160 at a slower speed and with a lower amplitude than pressure waves that do not travel through the noise-filtering portion 112 and the air pocket 120. This is because air in the air pocket 120 has a different density and, thus, a different impedance than the fluid that travels through the delivery portion 104 of the tube 102. When a pressure wave moves from a first material with a first impedance to a second material with a second, different, impedance, some of the pressure wave is scattered through the second material and some of the pressure wave is reflected back towards the first material. The amount of the wave that is scattered through the second material is inversely related to the difference between the first and second impedances. As a result, the air pocket 120, which has a much lower impedance than most fluids, reflects a lot of the pressure wave that is transmitted by the fluid in the delivery portion 104 towards the pressure transducer 160.

Notably, FIGS. 3A and 3B show that the air pocket 120 is surrounded by a first gel layer 116 and a second gel layer 118. These gel layers 116, 118 ensure that the pressure wave transmitted by the fluid in delivery portion 104 still reach the pressure transducer 160 instead of being completely reflected by the air pocket 120. Without the gel layers 116, 118, a pressure wave transmitted by the fluid in the delivery portion 104 directly into the air pocket 120 would be almost entirely reflected back into the fluid because the impedance difference between air and fluid is too large. Similarly, a pressure wave that travels through the air pocket 120 towards the pressure transducer 160 would be almost entirely reflected back into the air pocket 120 because the impedance difference between air and solids is too large. The gel that makes up the gel layers 116, 118 has an impedance that is greater than that of the air in the air pocket 120, but less than that of the fluid in the delivery portion 104 and the solid materials that make up the pressure transducer 160. This gradual change in impedance facilitates transmission of the pressure wave through the noise-filtering portion 112.

Specifically, the presence of the first gel layer 116 between the delivery portion 104 and the air pocket 120 ensures that the pressure wave is not entirely reflected by the air pocket because the layers create an incremental decrease in the impedance of the materials that the pressure wave must travel through on the way to the pressure transducer 160. Similarly, the presence of the second gel layer 118 between the air pocket 120 and the pressure transducer 160 ensures that the pressure wave is not entirely reflected by the pressure transducer 160. The layered structure (gel layer 116, air pocket 120, and gel layer 118) of the noise-filtering portion 112 of the tube 102 has an impedance arrangement that results in only some of the pressure wave being reflected at each layer. Because some of the pressure wave is reflected at each layer, only the strongest pressure waves (those with the highest amplitudes and the fastest speeds) transmitted from the fluid in the delivery portion 104 are received by the pressure transducer 160. Accordingly, the signal produced by the pressure transducer 160 of the noise-filtering fluid measurement system 100 is less noisy than a signal produced by a pressure transducer without any noise-filtering mechanism.

While the figures illustrate a noise-filtering portion 112 with a three-layer noise-filtering structure of the first gel layer 116, the air pocket 120, and the second gel layer 118, other embodiments of the layered noise-filtering structure are also possible to produce signals with varying levels of noise by changing the impedances of the materials in the structure. For example, the layered structure can have more than one gel layer on each side of the air pocket, and each gel layer can have a different impedance. The layered structure can be symmetrical, as shown, or asymmetrical (e.g., with more gel layers between the air pocket and the pressure transducer than gel layers between the fluid and the air pocket to account for the greater impedance difference between air and solid than air and fluid). The layered structure can also have multiple air pockets or even have pockets filled with different gases of different impedances.

FIGS. 3A and 3B also illustrate the positioning of the pressure transducer 160 relative to the noise-filtering portion 112. As described above with respect to FIGS. 2A and 2B, the noise-filtering portion 112 extends along a second longitudinal axis that is transverse to the first longitudinal axis of the delivery portion 104. The pressure transducer 160 is coupled to the mount 150 and positioned on the mount 150 such that the pressure transducer 160 aligns with the second longitudinal axis of the noise-filtering portion 112 when the tube 102 is coupled to the mount 150.

The distal opening 114 of the noise-filtering portion 112 can couple with the pressure transducer 160 in multiple ways. In the embodiment shown in FIG. 3A, the distal opening 114 surrounds the pressure transducer 160 such that the pressure transducer 160 is inside the second gel layer 118. The embodiment in FIG. 3B shows that the distal opening 114 is adjacent (or proximal to) the pressure transducer 160, such that the second gel layer 118 is also adjacent (or proximal to) the pressure transducer 160. Other embodiments of the noise-filtering fluid measurement system 100 have the pressure transducer 160 partially outside of the second gel layer 118 and partially within the second gel layer 118.

In some embodiments, membranes (e.g., a bellows or thin films) made of a flexible material that moves readily with the gel in response to receiving a pressure wave can separate the gel from the adjacent materials. For example, the first gel layer 116 can have a membrane that separates the gel in the first gel layer 116 from the fluid in the delivery portion 104. Similarly, the second gel layer 118 can have a membrane that separates the gel in the second gel layer 118 from the pressure transducer 160. The membranes can either be attached to the gel layers 116, 118, or be attached to the tube 102. For instance, the membrane that separates the gel in the first gel layer 116 from the fluid in the delivery portion 104 can be formed on the outside (here, the proximal side) of the first gel layer 116 or attached to the lateral opening 110 of the tube 102. Likewise, the membrane that separates the gel in the second gel layer 118 from the pressure transducer 160 can be formed on the outside (here, the distal side) of the second gel layer 118 or attached to the distal opening 114. Optionally, the gel layers 116, 118 have membranes that separate the gel from the air in the air pocket 120.

FIGS. 4A and 4B illustrate additional embodiments of the mount of the noise-filtering fluid measurement system, in accordance with some embodiments described herein. FIG. 4A shows a noise-filtering fluid measurement system 100′ with a mount 150′ having engagement features 152′ in the form of clamps that protrude from the surface of the mount 150′. The clamps 152′ grip the cylindrical surface of the delivery portion 104 of the tube 102. The clamps 152′ can be positioned at the edges of the mount 150′ (i.e., closer to the ends 106, 108 of the delivery portion 104), adjacent to the noise-filtering portion 112, or anywhere in between. The clamps 152′ can also be different sizes. For example, the clamps 152′ can be very narrow (e.g., when placed near the ends 106, 108 of the delivery portion) or can extend along the majority of the length of the delivery portion 104.

FIG. 4B shows a noise-filtering fluid measurement system 100″ with a mount 150″ having engagement features 152″ in the form of a tunnel or an interior channel. The mount 150″ has a smooth outer surface and a tunnel 152″ extending throughout. The tunnel 152″ is sized and shaped to fit the tube 102.

### Illustration of Subject Technology As Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A noise-filtering fluid delivery tube, the tube comprising: a delivery portion comprising two ends and a lateral opening between the two ends, wherein the delivery portion is configured to transport fluid from a fluid source to a patient; and a noise-filtering portion that is transverse to the delivery portion and intersects the delivery portion at the lateral opening, wherein the noise-filtering portion comprises a first gel layer adjacent to the lateral opening, a second gel layer adjacent to a distal opening opposite the lateral opening, and an air pocket between the first and second gel layers.

Clause 2. The tube of Clause 1, wherein the delivery portion and the noise-filtering portion each comprise a hollow cylinder, the noise-filtering portion being shorter than the delivery portion.

Clause 3. The tube of Clause 1, wherein each of the two ends comprises a needleless fluid connector.

Clause 4. The tube of Clause 3, wherein each of the needleless fluid connectors comprises a male luer lock or a female luer lock.

Clause 5. The tube of Clause 1, wherein the lateral opening comprises a lateral membrane adjacent to the first gel layer, the lateral membrane being configured to separate the fluid in the delivery portion from the first gel layer.

Clause 6. The tube of Clause 1, wherein the distal opening of the noise-filtering portion is configured to couple with a pressure transducer.

Clause 7. The tube of Clause 6, wherein the distal opening comprises a distal membrane adjacent to the second gel layer, wherein the distal membrane is configured to separate the second gel layer from the pressure transducer.

Clause 8. The tube of Clause 6, wherein the second gel layer of the noise-filtering portion is configured to surround the pressure transducer.

Clause 9. The tube of Clause 6, wherein the first gel layer is configured to separate the fluid from the air pocket, and the second gel layer is configured to separate the air pocket from the pressure transducer.

Clause 10. The tube of Clause 1, wherein gel in the first and second gel layers comprises a first impedance and air in the air pocket comprises a second impedance, the first impedance being greater than the second impedance, but less than an impedance of the fluid being transported through the delivery portion.

Clause 11. A noise-filtering fluid delivery tube, the tube comprising: a delivery portion configured to fluidly connect a fluid source and a patient; and a noise-filtering portion transverse to the delivery portion, the noise-filtering portion comprising a gel filling and an air pocket positioned within the gel filling.

Clause 12. The tube of Clause 11, wherein the gel filling comprises a first gel layer adjacent to the delivery portion, and the tube further comprises a first membrane between the first gel layer and the delivery portion.

Clause 13. The tube of Clause 11, wherein the gel filling is configured to abut a pressure transducer.

Clause 14. The tube of Clause 13, wherein the gel filling comprises a second gel layer that is configured to abut the pressure transducer, and the noise-filtering portion further comprises a second membrane configured to separate the second gel layer from the pressure transducer.

Clause 15. The tube of Clause 11, wherein the gel filling is configured to surround a pressure transducer.

Clause 16. A noise-filtering fluid measurement system, the system comprising: a fluid delivery tube comprising: a delivery portion configured to transport fluid from a fluid source to a patient, wherein the delivery portion extends along a first longitudinal axis and comprises a lateral opening; and a noise-filtering portion extending from the delivery portion at the lateral opening along a second longitudinal axis that is transverse to the first longitudinal axis, wherein the noise-filtering portion comprises a distal opening opposite the lateral opening, a gel filling between the lateral and distal openings, and an air pocket within the gel filling; a mount comprising a tube engagement feature, wherein the fluid delivery tube is coupled to the mount via the tube engagement feature; and a pressure transducer coupled to the mount, wherein the pressure transducer is aligned with the second longitudinal axis of the noise-filtering portion.

Clause 17. The system of Clause 16, wherein the gel filling surrounds the pressure transducer.

Clause 18. The system of Clause 16, wherein the gel filling comprises a first gel layer in between the lateral opening and the air pocket and a second gel layer in between the air pocket and the distal opening.

Clause 19. The system of Clause 16, wherein gel in the gel filling comprises a first impedance and air in the air pocket comprises a second impedance, the first impedance being greater than the second impedance, but less than an impedance of the fluid being transported through the delivery portion.

Clause 20. The system of Clause 16, wherein the tube engagement feature comprises one or more of a channel, a plurality of clamps, and a cavity.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A noise-filtering fluid delivery tube, the tube comprising:
a delivery portion comprising two ends and a lateral opening between the two ends, wherein the delivery portion is configured to transport fluid from a fluid source to a patient; and
a noise-filtering portion that is transverse to the delivery portion and intersects the delivery portion at the lateral opening, wherein the noise-filtering portion comprises a first gel layer adjacent to the lateral opening, a second gel layer adjacent to a distal opening opposite the lateral opening, and an air pocket between the first and second gel layers.

2. The tube of claim 1, wherein the delivery portion and the noise-filtering portion each comprise a hollow cylinder, the noise-filtering portion being shorter than the delivery portion.

3. The tube of claim 1, wherein each of the two ends comprises a needleless fluid connector.

4. The tube of claim 3, wherein each of the needleless fluid connectors comprises a male luer lock or a female luer lock.

5. The tube of claim 1, wherein the lateral opening comprises a lateral membrane adjacent to the first gel layer, the lateral membrane being configured to separate the fluid in the delivery portion from the first gel layer.

6. The tube of claim 1, wherein the distal opening of the noise-filtering portion is configured to couple with a pressure transducer.

7. The tube of claim 6, wherein the distal opening comprises a distal membrane adjacent to the second gel layer, wherein the distal membrane is configured to separate the second gel layer from the pressure transducer.

8. The tube of claim 6, wherein the second gel layer of the noise-filtering portion is configured to surround the pressure transducer.

9. The tube of claim 6, wherein the first gel layer is configured to separate the fluid from the air pocket, and the second gel layer is configured to separate the air pocket from the pressure transducer.

10. The tube of claim 1, wherein gel in the first and second gel layers comprises a first impedance and air in the air pocket comprises a second impedance, the first impedance being greater than the second impedance, but less than an impedance of the fluid being transported through the delivery portion.

11. A noise-filtering fluid delivery tube, the tube comprising:
a delivery portion configured to fluidly connect a fluid source and a patient; and
a noise-filtering portion transverse to the delivery portion, the noise-filtering portion comprising a gel filling and an air pocket positioned within the gel filling.

12. The tube of claim 11, wherein the gel filling comprises a first gel layer adjacent to the delivery portion, and the tube further comprises a first membrane between the first gel layer and the delivery portion.

13. The tube of claim 11, wherein the gel filling is configured to abut a pressure transducer.

14. The tube of claim 13, wherein the gel filling comprises a second gel layer that is configured to abut the pressure transducer, and the noise-filtering portion further comprises a second membrane configured to separate the second gel layer from the pressure transducer.

15. The tube of claim 11, wherein the gel filling is configured to surround a pressure transducer.

16. A noise-filtering fluid measurement system, the system comprising:
a fluid delivery tube comprising:
a delivery portion configured to transport fluid from a fluid source to a patient, wherein the delivery portion extends along a first longitudinal axis and comprises a lateral opening; and
a noise-filtering portion extending from the delivery portion at the lateral opening along a second longitudinal axis that is transverse to the first longitudinal axis, wherein the noise-filtering portion comprises a distal opening opposite the lateral opening, a gel filling between the lateral and distal openings, and an air pocket within the gel filling;
a mount comprising a tube engagement feature, wherein the fluid delivery tube is coupled to the mount via the tube engagement feature; and
a pressure transducer coupled to the mount, wherein the pressure transducer is aligned with the second longitudinal axis of the noise-filtering portion.

17. The system of claim 16, wherein the gel filling surrounds the pressure transducer.

18. The system of claim 16, wherein the gel filling comprises a first gel layer in between the lateral opening and the air pocket and a second gel layer in between the air pocket and the distal opening.

19. The system of claim 16, wherein gel in the gel filling comprises a first impedance and air in the air pocket comprises a second impedance, the first impedance being greater than the second impedance, but less than an impedance of the fluid being transported through the delivery portion.

20. The system of claim 16, wherein the tube engagement feature comprises one or more of a channel, a plurality of clamps, and a cavity.
